(19) United States
(12) Patent Application Publication
(10) Pub. No.: US 2025/0262463 A1
(43) Pub. Date: Aug. 21, 2025

(54) MODULATION METHOD FOR ULTRASONIC OUTPUT PULSES, CONTROLLER AND TREATMENT APPARATUS

(71) Applicant: SHENZHEN PENINSULA MEDICAL GROUP, Shenzhen (CN)

(72) Inventors: Yanan LI, Shenzhen (CN); Xingli LI, Shenzhen (CN); Xiaobing LEI, Shenzhen (CN); Yi DING, Shenzhen (CN)

(73) Assignee: SHENZHEN PENINSULA MEDICAL GROUP, Shenzhen (CN)

(21) Appl. No.: 19/204,125

(22) Filed: May 9, 2025

Related U.S. Application Data
(63) Continuation of application No. PCT/CN2023/082819, filed on Mar. 21, 2023.

(30) Foreign Application Priority Data
Sep. 15, 2022 (CN) 202211122044.2

(51) Int. Cl. A61N 7/00

(52) U.S. Cl. CPC A61N 7/00

(57) ABSTRACT

Disclosed are a modulation method for ultrasonic output pulses, a controller and a treatment apparatus. The modulation method for ultrasonic output pulses includes: obtaining an output frequency of the first preset pulse, and modulating a to-be-modulated pulse according to the output frequency of the first preset pulse to obtain a first pulse train; obtaining a period length of the second preset pulse, and dividing the first pulse train into N of the second pulse trains according to the period length of the second preset pulse; and controlling a transducer to sequentially output the N of the second pulse trains.

### CROSS-REFERENCE TO RELATED APPLICATIONS

This application is a continuation application of International Application No. PCT/CN2023/082819, filed on Mar. 21, 2023, which claims priority to Chinese Patent Application No. 202211122044.2, filed on Sep. 15, 2022. The disclosures of the above-mentioned applications are incorporated herein by reference in their entireties.

### TECHNICAL FIELD

The present application relates to the field of ultrasonic treatment apparatus, and in particular to a modulation method for ultrasonic output pulses, a controller and an ultrasonic treatment apparatus.

### BACKGROUND

A High intensity focused ultrasound (HIFU) device is different from a laser device and a radio frequency (RF) device, and it concentrates energy on the selected part in a non-invasive manner without causing any damage to the skin surface, that is, focusing the outputted ultrasound energy on a predetermined treatment area to generate heat, thereby inducing a sharp raising temperature of the treatment area. With this temperature rise function, no side effects are left on various affected areas, and adipose cells are treated to undergo coagulation and ablation until necrosis.

The ultrasonic device emits the focused ultrasonic energy in a form of pulses, and each ultrasonic pulse forms a heat diffusion zone in the target tissue. Most of the ultrasonic energy outputted by the ultrasonic pulse is concentrated on the central area of the heat diffusion zone, and the energy dispersal range is narrow, which affects the treatment effect of the entire treatment area. In addition, due to the excessive concentration of energy, the central temperature gets high sharply, which easily causes patients to feel tingling with poor user experience.

### SUMMARY

The main purpose of the present application is to propose a modulation method for ultrasonic output pulses, a controller and a treatment apparatus for ultrasonic output pulses, which aims to reduce pain and improve the treatment effect by improving the energy uniformity of the treatment area.

To achieve the above purpose, the present application proposes a modulation method for ultrasonic output pulses, including the following steps:
step S10, obtaining an output frequency f2 of a first preset pulse, and modulating a to-be-modulated pulse according to the output frequency f2 of the first preset pulse to obtain a first pulse train;
step S20, obtaining a period length t0 of a second preset pulse, and dividing the first pulse train into N of the second pulse trains according to the period length t0 of the second preset pulse; and step S30, controlling a transducer to sequentially output the N of the second pulse trains.

In an embodiment, the step S20 further includes:
step S21, obtaining a pulse width of a period of the second preset pulse and an Off time of the period of the second preset pulse; and
step S22, determining the period length t0 of the second preset pulse according to the pulse width of the period of the second preset pulse and the Off time of the period of the second preset pulse.

In an embodiment, the pulse width of the period of the second preset pulse is less than a constant time for human pain perception.

In an embodiment, a number of pulse widths of the N of the second preset trains is identical with a number of pulse widths within a first pulse train period TO.

In an embodiment, the modulation method for ultrasonic output pulses further includes:
adjusting a duty ratio of each of the pulse trains and/or a voltage amplitude of each of the pulse trains to control an output power of each of the pulse trains.

In an embodiment, the adjusting the duty ratio of each of the pulse trains further includes:
adjusting the pulse width and/or an Off time of each of the pulse trains to control the duty ratio of each of the pulse trains.

In an embodiment, the step S30 further includes:
controlling a treatment tip to vibrate slightly along a direction parallel with a skin surface while controlling the transducer to sequentially output the N of the second pulse trains.

The present application proposes a controller including a memory and a processor, the memory stores a control program for ultrasonic output pulses, and the modulation method for ultrasonic output pulses as described above are implemented when the control program for ultrasonic output pulses is executed by the processor.

The present application proposes an ultrasonic treatment apparatus, including:
a treatment tip provided with a transducer inside;
an ultrasonic generating unit for modulating ultrasonic pulses outputted by the transducer; and
the controller as described above connected to the ultrasonic generating unit and configured to control the ultrasonic generating unit to operate to modulate pulse trains outputted by the transducer;
the controller is further configured to control the treatment tip to vibrate slightly along a direction parallel with a skin surface, and control the transducer to output the pulse trains.

In an embodiment, the ultrasonic treatment apparatus further includes an ultrasonic handpiece, and the treatment tip is provided at the ultrasonic handpiece; the ultrasonic handpiece includes a point-type handpiece and a linear-type handpiece; the linear-type handpiece is configured to apply multiple focuses of the ultrasonic energy in line at one shot, being capable of discretely moving at a preset interval along a skin surface of the treatment area; and the point-type handpiece is configured to apply one focus of the ultrasonic energy at one shot, being capable of continuously sliding and overlapping for a preset time along the skin surface of the treatment area.

The modulation method for ultrasonic output pulses of the present application includes the following steps: step S10, obtaining an output frequency f2 of a first preset pulse, and modulating a to-be-modulated pulse according to the output frequency f2 of the first preset pulse to obtain a first pulse train; step S20, obtaining a period length t0 of a second preset pulse, and dividing the first pulse train into N of the second pulse trains according to the period length to of the second preset pulse; and step S30, controlling a transducer to sequentially output the N of the second pulse trains. When working, obtaining a to-be-modulated pulse, dividing the obtained to-be-modulated pulse into N of the pulse trains according to the preset pulse output frequency f2 and the preset pulse train period length t0, and outputting N of the pulse trains sequentially according to N of the periods. Compared with the output of a single pulse, the energy output by multiple pulse trains is not an instantaneous burst, so the temperature of the central area of the heat diffusion zone formed will not be too high, and the range of dispersal to the surroundings will be relatively large. After modulating the pulse, the pain can be effectively reduced and the comfort can be improved, the heat diffusion zone formed is larger at the same time, so that the energy of the entire treatment area is more uniform, thus improving the treatment effect. The present application reduces pain and improves treatment effects by increasing uniformity of ultrasound energy applied in the treatment area.

### BRIEF DESCRIPTION OF THE DRAWINGS

In order to explain the embodiments of the present application or the technical solutions in the existing technology more clearly, the accompanying drawings needed to be used in the description of the embodiments or the existing technology will be briefly introduced below. Obviously, the accompanying drawings in the following description are only some embodiments of the present application, other accompanying drawings can be obtained based on the provided accompanying drawings without exerting creative efforts for those skilled in the art.

FIG. 1 is a flowchart of a modulation method for ultrasonic output pulses according to an embodiment of the present application.

FIG. 2 is a flowchart of an embodiment of step S20 in FIG. 1.

FIG. 3 is a waveform diagram of a modulation pulse of a modulation method for ultrasonic output pulses according to an embodiment of the present application.

FIG. 4 is a comparison diagram of a heat diffusion zone formed by a single ultrasound pulse and a heat diffusion zone formed by multiple pulse trains.

FIG. 5 is a waveform diagram of adjusting pulse train power according to the present application.

### DETAILED DESCRIPTION OF THE EMBODIMENTS

The technical solutions in the embodiments according to the present application will be clearly and completely described below in conjunction with the accompanying drawings in the embodiments according to the present application. Obviously, the described embodiments are only a part of the embodiments according to the present application, and not all of the embodiments. Based on the embodiments in the present application, all other embodiments obtained by those skilled in the art without making creative labor fall within the scope of the present application. It should be noted that if the embodiments of the present application involve directional indications (such as up, down, left, right, front, back . . . ), such directional indications are only used to explain the relative positional relationship, movement and so on between various components in a specific posture (as shown in the accompanying drawings). If the specific posture changes, the directional indication will also change accordingly.

In addition, if there are descriptions involving “first”, “second” or the like in the embodiments of the present application, the descriptions of “first”, “second” or the like are only for descriptive purposes and cannot be understood as indicating or implying the relative importance or implicitly indicating the quantity of the technical features indicated. Therefore, features defined as “first” and “second” may explicitly or implicitly include at least one of these features. In addition, the technical solutions between various embodiments can be combined with each other, but it is based on that those of ordinary skill in the art can realize. When the combination of technical solutions is contradictory or cannot be realized, it should be considered that such combination of technical solutions does not exist and is not within the protection scope claimed by the present application.

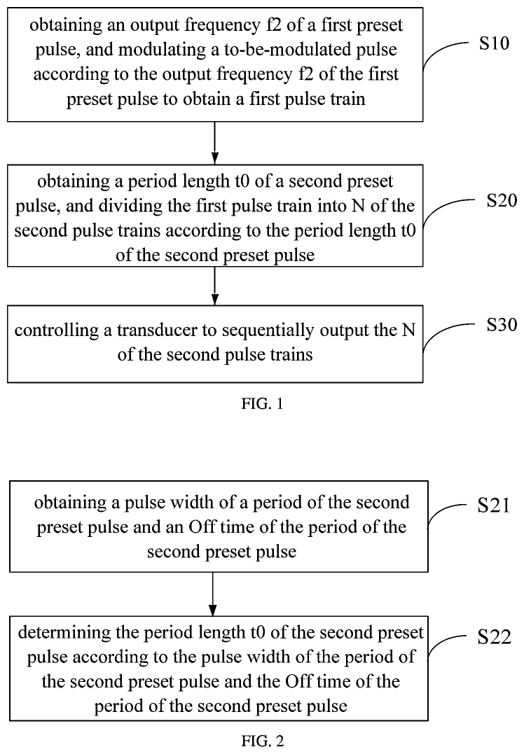

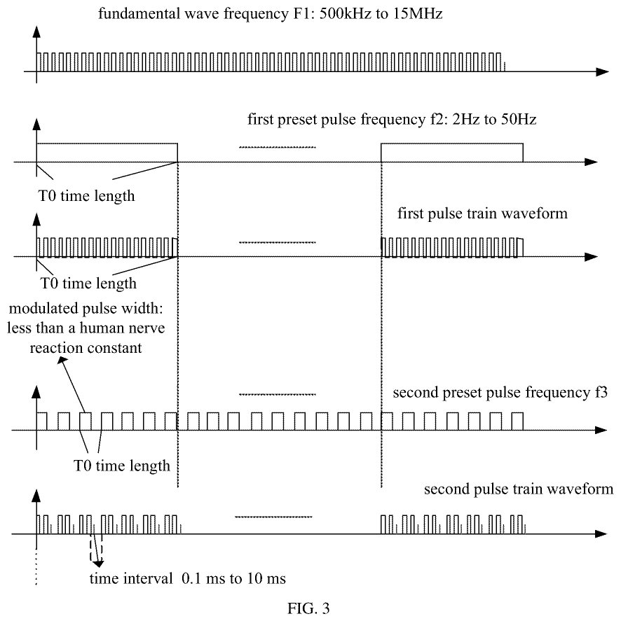

Referring to FIG. 1 and FIG. 3, in an embodiment of the present application, the modulation method for ultrasonic output pulses includes the following steps.
Step S10, obtaining an output frequency f2 of a first preset pulse, and modulating a to-be-modulated pulse according to the output frequency f2 of the first preset pulse to obtain a first pulse train.
Step S20, obtaining a period length t0 of a second preset pulse, and dividing the first pulse train into N of the second pulse trains according to the period length t0 of the second preset pulse.
Step S30, controlling a transducer to sequentially output the N of the second pulse trains.

The ultrasonic treatment apparatus achieves the treatment effect by outputting ultrasound pulses to the subcutaneous tissue. The ultrasonic pulse generates a heat diffusion zone in the target tissue, so that the focused ultrasound pulse acts on the subcutaneous tissue, and the treatment range of the ultrasonic treatment apparatus is diffused by diffusing the area where the focused ultrasound pulse applies on. Each ultrasonic pulse forms a heat diffusion zone in the target tissue, and most of ultrasound pulse of the output pulse is concentrated in the central area of the heat diffusion zone, and the energy dispersal range is narrow, which affects the treatment effect of the entire treatment area.

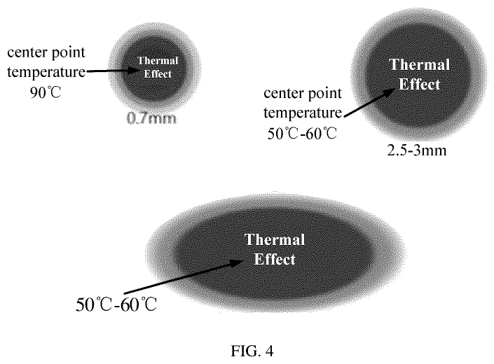

Referring to FIG. 4, if each ultrasonic pulse is output in the form of a single ultrasound pulse, the output ultrasonic pulse instantly hits the tissue at the focal point position, and the tissue at the focal point will heat up sharply, and the center temperature can instantly reach 90° C. At the same time, the heat will diffuse to the surroundings to form a heat diffusion zone. Because the energy is relatively concentrated, the heat diffusion zone is narrow and the diameter does not exceed 1 mm. The instantaneous high temperature generated in the tissue at the focal point will cause the user to feel tingling, and because the heat diffusion zone is very narrow and the energy distribution is uneven, the treatment effect of ultrasonic treatment is poor.

In this embodiment, a to-be-modulated pulse is modulated into multiple pulse trains for outputting, that is, a single ultrasound pulse (one ultrasound pulse) is modulated into pulse trains for output, and the total energy of the pulse trains is identical with the energy of a single ultrasound pulse, that is, when multiple small pulses are sequentially hit on the tissue at the focal point, the tissue at the focal point heats up under the superposition of energy, and the heat also diffuses to the surroundings at the same time.

The working process of modulating the pulse is specifically as follows: firstly, modulating the ultrasonic operating frequency of the pulse generated by the ultrasonic generation source, and obtaining the pulse train after the first modulation, which is recorded as the first pulse train; dividing the first pulse train into the N of the second pulse trains.

The working process of executing step S10 is as follows: performing frequency modulation on the pulses generated by the ultrasonic generation source, assuming that the ultrasonic operating frequency of the pulse generated by the ultrasonic generation source is f1, and the output frequency of the modulated pulse train is f2. In general, the frequency f1 of the pulse generated by the ultrasonic generation source is too large and cannot be directly output as a ultrasound pulse. The value of the operating frequency f2 required by the ultrasound pulse is much smaller than the value of f1 (f1 is generally taken between 500 kHz and 15 MHz, and f2 is generally taken between 2 Hz and 50 Hz). Taking FIG. 3 as an example, which explains the specific working process of modulation, the modulation process can be similar to the pulse generated by the ultrasonic generation source flowing through a “switch”. For a part of the pulses generated by the original ultrasonic generation source, the “switch” is turned on, and the pulse is still output in the form of a fundamental wave. For another part of the pulses generated by the original ultrasonic generation source, the “switch” is turned off and the pulse stops outputting. It can be understood that the to-be-modulated pulse is always output in the form of a fundamental wave, and the first pulse train after the modulation is output in the form of a fundamental wave for a period of time, and no pulse is output for another period of time. Therefore, the frequency of the first pulse train is smaller than the frequency of the to-be-modulated pulse.

The working process of executing step S20 is as follows: dividing the first pulse train into the N of the second pulse trains, assuming that the pulse period output by the first pulse train is T0, that is, the output first pulse train is output in the form of fundamental wave in a T0 time length. Then, modulating each pulse of T0 time length to t0 to output; assuming that modulated pulses are N pulses in total, and the period of each second pulse train is t0, T0=N*t0. It should be noted that the period of each of the pulse trains includes pulse width and pulse interval. The pulse interval of the pulse train is the time when there is no pulse train output, and the pulse width of the pulse train is the time when there is pulse train output; in the pulse width of the pulse train, it is still output in the form of fundamental wave, rather than in the form of high potential.

Since there is an Off time between multiple pulse trains formed after modulation, the output energy is not an instantaneous burst, so the temperature of the heat diffusion zone in the center area will not be too high, and the range of dispersal to the surroundings will be relatively large. Using focused ultrasound pulses to act on subcutaneous tissue, a temperature between 50° C. and 60° C. can achieve a better treatment effect. Too high a temperature will increase the risk of burns or cause unnecessary damage that is not conducive to recovery. At the same time, too high an instantaneous temperature will bring a strong tingling sensation. After pulse modulation, it can effectively reduce pain and improve comfort, the heat diffusion zone formed is larger at the same time, thus making the energy of the entire treatment area more uniform, and improving the treatment effect.

The modulation method for ultrasonic output pulses of the present application includes the following steps: step S10, obtaining the output frequency f2 of the first preset pulse, and modulating a to-be-modulated pulse according to the output frequency f2 of the first preset pulse to obtain a first pulse train; step S20, obtaining the period length t0 of the second preset pulse, and dividing the first pulse train into N of the second pulse trains according to the period length t0 of the second preset pulse; and step S30, controlling a transducer to sequentially output the N of the second pulse trains. Compared with the output of a single pulse, the energy output by multiple pulse trains is not an instantaneous burst, so the temperature of the central area of the heat diffusion zone formed will not be too high, and the range of dispersal to the surroundings will be relatively large. After modulating the pulse, the pain can be effectively reduced and the comfort can be improved, the heat diffusion zone formed is larger at the same time, so that the energy of the entire treatment area is more uniform, thus improving the treatment effect. The present application reduces pain and improves treatment effects by increasing energy uniformity in the treatment area.

Referring to FIG. 2, in an embodiment of the present application, the step S20 further includes the following steps.
Step S21, obtaining a pulse width of a period of the second preset pulse and an Off time of the period of the second preset pulse.
Step S22, determining the period length t0 of the second preset pulse according to the pulse width of the period of the second preset pulse and the Off time of the period of the second preset pulse. In this embodiment, the length of the period of a second preset pulse includes the pulse width and the Off time. It should be noted that when the 1-pulse option is selected, the ultrasonic treatment apparatus has an Off time where no pluse waveform is outputted after a pulse width where pulse waveform is output during the period for outputting the second preset pluse. It should be noted that the pulse interval of the pulse train is the time when there is no pulse train output, and the pulse width of the pulse train is the time when there is pulse train output. It should be noted that, the output within the pulse width of the pulse train maintains the form of the fundamental wave, rather than a high-level voltage applied, in which, the fundamental wave refers to the equivalent continuous waveform reconstructed through PWM modulation and filtering, and the high-level voltage indicates discrete digital-level output (e.g., fixed high voltage without modulation).

Referring to FIG. 3, in an embodiment of the present application, the pulse width of the period of the second preset pulse is less than a constant time for human pain perception.

In this embodiment, the time for human pain perception is a time of a process of receiving the stimulation of information and then reacting. The human body can feel the stimulation only after the nerve reflex arc is completed. The constant time to for human pain perception is in a range of 0.5 ms and 2 ms. The N of the pulse train periods are all less than the constant time t0 for human pain perception. The specific value of t0 is related to the position of the human nerve, and the constant time t0 for pain perception of different parts of the human body is different, so the pulse width of the period can be preset according to the treatment area. For example, when the constant time t0 for human pain perception corresponding to a certain treatment area is 1 ms, the pulse width of the period can be set to less than 1 ms.

The action time of a single pulse train is less than the constant time for human pain perception, the energy is applied before the nerve reflex arc is completed, and the output of the ultrasound pulse is completed before the neuron receives the stimulation signal, which can effectively reduce the stimulation to the skin during treatment.

Referring to FIG. 3, in an embodiment of the present application, a number of pulse widths of the N of the second preset pulse periods t0 is identical with a number of pulse widths of a first pulse train period T0.

In this embodiment, in the process of the first pulse train period T0 being divided into N of the second pulse trains, in the process of the first pulse train period T0 being divided into N of the second pulse trains, modulating the Off time of the to-be-modulated pulse preferentially. The pulse width outputted by the pulse train is not changed, that is, the pulse energy outputted by the first pulse train within the T0 time length is consistent with the number of the pulse energy outputted by the N of the second pulse trains within the t time length. The pulse width of the first pulse train and the pulse width of the second pulse train still output the ulstrasound energy in the form of fundamental wave.

Referring to FIG. 3, the Off time of each preset second pulse train is generally taken in a range of 0.1 ms to 10 ms. It should be noted that the Off time of the preset second pulse train refers to the Off time between two adjacent pulse widths in the output waveform of the second pulse train. In one period t0 of the second pulse train, one pulse width and one Off time are included. The pulse width of the second pulse train in the period to outputs energy in the form of fundamental wave, and no energy is outputted in the Off time of the period to.

Referring to FIG. 4, in an embodiment of the present application, the step S30 also includes the following step:

controlling a treatment tip to vibrate slightly along a direction parallel with a skin surface while controlling the transducer to sequentially output the N of the second pulse trains according to N of the periods.

In this embodiment, a handpiece is provided with a drive unit for driving the treatment tip to vibrate along a direction parallel with the treatment surface to output multiple modulated pulse trains, and the multiple modulated pulse trains may have better treatment effects.

In a static state, the heat diffusion zone formed by the ultrasound pulse outputted by the transducer is relatively small, and the temperature of the central area is relatively high; the heat diffusion zone formed by the ultrasound pulse outputted during transducer's movement is relatively large, and the temperature of the central area of the heat diffusion zone is lower than the central temperature of the heat diffusion zone formed in the static state, which can effectively reduce pain.

When working, the treatment end (ultrasound window) of the treatment tip is close to the skin surface. When the controller modulates the outputted pulse train, it controls the treatment tip to vibrate slightly along a direction parallel with a skin surface, and outputs the ultrasound pulse train at the same time. The focal positions of outputted pulse train are constantly changed, under this circumstance, the heat diffusion zone formed is relatively small, and the temperature in the center area is relatively high. The temperature in the center area of the heat diffusion zone is lower than the center temperature of the heat diffusion zone formed in the static state, which can effectively reduce the pain. At the same time, since the area of the heat diffusion zone is larger, the treatment area formed is larger, which can play a massage role on the skin in the treatment area, thus effectively alleviating the pain, and promoting the blood circulation of the tissue in the treatment area, and helping the recovery of the tissue.

Ultrasound pulse uses a point-type pulse outputted method. Each shot, the ultrasound pulse is focused at one point, the energy focused points are successively formed in the tissue during multiple shots. Since an Off time is existed between multiple pulse trains after modulation, the outputted energy is not an instantaneous burst, so the temperature of the heat diffusion zone in the center area will not be too high, and the range of dispersal to the surroundings will be relatively large. Focused ultrasound pulses is used to act on subcutaneous tissue, a temperature between 50° C. and 60° C. can achieve a better treatment effect. The temperature being too high will increase the risk of burns or cause unnecessary damage that is not beneficial to recovery. At the same time, excessive temperature happens instantaneously will bring a strong tingling sensation. After pulse modulation, it can effectively reduce pain and improve comfort, the heat diffusion zone formed is larger at the same time, thus making the energy of the entire treatment area more uniform, and improving the treatment effect.

By controlling the sliding of the handpiece to drive the transducer to change focused positions formed by the outputted pulse train; in the process of controlling the sliding of the handpiece, the focuses outputted by the transducer are sliding according to the sliding movement of the handpiece, finally forming a spherical-shaped heat diffusion zone changed from the sphere-shaped heat diffusion zone. While the sliding of the handpiece is controlled, the treatment tip is controlled to vibrate side by side along the skin surface, so that the ultrasound pulse emitted by the transducer is slightly offset compared with the ulstrasound pulse outputted by the sliding movement of the handpiece, and the heat diffusion zone is changed from the originally spherical-shape to an irregular ellipsoid shape, thus further increasing the range of the heat diffusion zone and improving the treatment effect. The vibration movement of the treatment tip is combined with the sliding movement of the handpiece which avoids two adjacent ultrasound pulses to be focused at the same position of the tissue, thus reducing the risk of burns.

In FIG. 4, the vibration movement of the treatment tip is combined with a sliding movement of the handpiece, thus the focused area is changed from a circle treatment area to an irregular elliptical treatment area, the heat diffusion zone has larger range, the energy distribution is more uniform, and the treatment effect is better.

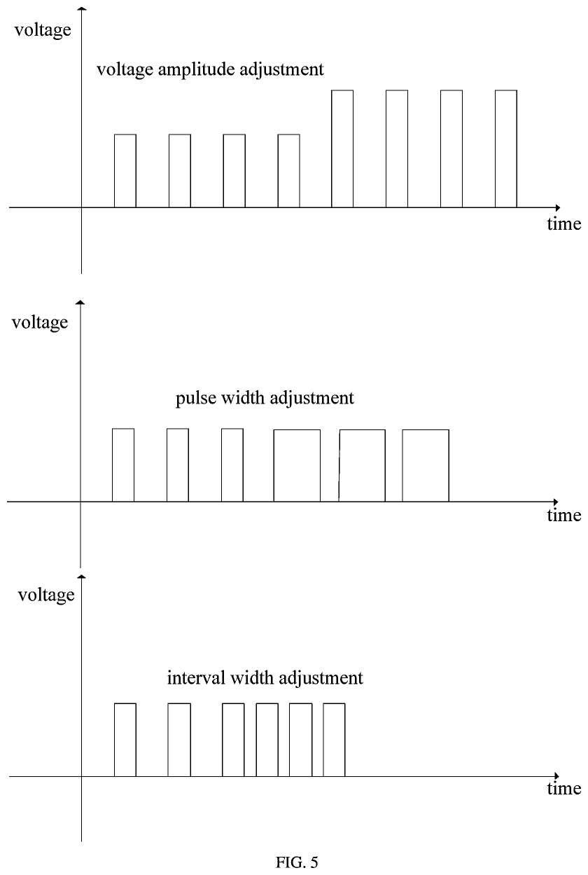

Referring to FIG. 5, in an embodiment of the present application, controlling the duty ratio of each of the pulse trains further includes the following step:
adjusting a duty ratio of each of the pulse trains and/or a voltage amplitude of each of the pulse trains to control an output power of each of the pulse trains.

In this embodiment, the ultrasonic output frequency of the transducer is achieved by modulating its to-be-modulated pulse, which is specifically controlled by modulating the time and amplitude of the to-be-modulated pulse of the ultrasonic unit. For the duty ratio of the to-be-modulated pulse of the ultrasonic unit, the larger the output frequency of the corresponding to-be-modulated pulse of the ultrasonic unit is, the larger the amplitude of the to-be-modulated pulse is, and the larger the output frequency of the corresponding to-be-modulated pulse of the ultrasonic unit is.

Referring to FIG. 5, in an embodiment of the present application, controlling the duty ratio of each of the pulse trains further includes the following step:
adjusting the pulse width and/or an Off time of each of the pulse trains to control the duty ratio of each of the pulse trains.

In this embodiment, specifically, the size of the pulse width and/or the size of the Off time is/are controlled to control the size of the duty ratio of each of the pulse trains. In other word, the larger the modulated pulse width is, the larger the output duty ratio is, and the larger the Off time of the modulated pulse is, the smaller the output duty ratio is.

In an embodiment of the present application, before controlling the transducer to output pulses, the following steps are further included:
detecting whether a surface of the treatment tip is insufficient contact with the skin; and
when detecting that the surface of the treatment tip is insufficient contact with the skin, controlling the transducer to output pulses; when detecting that the surface of the treatment tip is not insufficient contact with the skin, outputting a corresponding alarm signal.

In this embodiment, before the output of the ultrasound pulse is controlled according to the detected speed, whether the surface of the treatment tip is insufficient contact with the skin firstly is detected; if it is not fitted, the speed feedback step is no longer performed. Since the ultrasound pulse outputted by the ultrasonic treatment transducer may cause skin burns when the surface of the treatment tip is insufficiently contacted with the skin, the speed feedback step is not executed, and a corresponding alarm signal is outputted to warn the operator.

In an embodiment of the present application, the modulation method for ultrasonic output pulses further includes the following steps:
detecting temperature of the skin surface;
when the detected temperature is higher than a preset temperature value, controlling the transducer to stop outputting the ultrasound pulse and outputting a corresponding alarm signal; when the detected temperature is lower than the preset temperature value, outputting an operation guidance signal, and controlling the transducer to continue outputting the ultrasound pulse.

In this embodiment, during the process of the ultrasonic treatment transducer the ultrasound pulse is outputted to the subcutaneous tissue, the temperature of the skin surface will also increase accordingly. When the detected temperature value is lower than the preset temperature value, the temperature of the skin surface being still in the safe temperature zone is determined at this time, and a guidance signal is outputted to warn that the operator can continue to perform ultrasound treatment on the area.

When the detected temperature value is higher than the preset temperature value, determining that the temperature of the skin surface is too high, and the ultrasonic treatment transducer stops outputting the ultrasound pulse to the subcutaneous tissue to avoid skin burns, and a corresponding alarm signal is outputted to warn the operator.

The present application proposes a controller.

In an embodiment of the present application, the controller includes a memory and a processor, the memory stores a control program for ultrasonic output pulses, and the modulation method for ultrasonic output pulses as described above are implemented when the control program for ultrasonic output pulses is executed by the processor.

The controller executes the steps of the control method for the ultrasonic output pulse as described above. The specific working steps of the control method for the ultrasonic output pulse refer to the above embodiment. Since the controller of the present application adopts all the technical solutions of all the above embodiments, it at least has all the beneficial effects brought by the technical solutions of the above embodiments, which will not be repeated here.

The present application proposes an ultrasonic treatment apparatus.

The ultrasonic treatment apparatus includes the controller as described in above embodiments. The specific structure of the controller refers to the above embodiments. Since the ultrasonic treatment apparatus of the present application adopts all the technical solutions of all the above embodiments, it at least has all the beneficial effects brought by the technical solutions of the above embodiments, which will not be repeated here.

In an embodiment, an ultrasonic treatment apparatus includes:
a treatment tip provided with a transducer inside;
an ultrasonic generating unit for modulating ultrasonic pulses outputted by the transducer; and
the controller as described above connected to the ultrasonic generating unit and configured to control the ultrasonic generating unit to operate to modulate pulse trains outputted by the transducer; and
the controller is further configured to control the treatment tip to vibrate slightly along a direction parallel with a skin surface, and control the transducer to output the pulse trains.

In an embodiment, the ultrasonic treatment apparatus further includes an ultrasonic handpiece, and the treatment tip is carried on the ultrasonic handpiece; the ultrasonic handpiece includes a point-type handpiece and a linear-type handpiece; the linear-type handpiece is configured to apply multiple focuses of the ultrasonic energy in line at one shot, being capable of discretely moving at a preset interval along a skin surface of the treatment area; and the point-type handpiece is configured to apply one focus of the ultrasonic energy at one shot, being capable of continuously sliding and overlapping for a preset time along the skin surface of the treatment area.

The operator moves the handpiece during treatment, keeps the ultrasonic window close to the skin and slides, and the ultrasound pulse is outputted in a pulsed manner. The focal positions of the ultrasound pulse are controlled by continuously moving the point-type handpiece or the linear-type handpiece to achieve the corresponding treatment effect. The ultrasonic transducer inside the treatment tip outputs focused ultrasound pulses, which manifests as heating effects to cause temperature risen on tissues, and the heat diffuses from the focal point to its surroundings to form a heat diffusion zone.

A vibration motor is provided on the treatment tip, and a driving control circuit for driving the vibration motor is provided in the controller. While the controller controls the transducer to output the ultrasound pulse, the driving control circuit also drives the vibration motor to work, so that the treatment tip vibrates slightly along a direction parallel with a skin surface, so that the action position of the ultrasound pulse is slightly offset, thus further diffusing the action range of the heat diffusion zone. A corresponding sensor, such as a temperature sensor, is provided on the vibration motor. The controller can control the operating frequency of the vibration motor according to the temperature detected by the temperature sensor to control the vibration power of the treatment tip, which is conductive to the heat diffusion zone.

The vibration motor is equipped with a set of adjustable eccentric blocks at both ends of the rotor shaft, the centrifugal force generated by the high-speed rotation of the shaft and the eccentric blocks is used to generate the exciting force, thereby the treatment tip is driven to vibrate slightly by itself. Since the treatment tip has a relatively small vibration power, a low-power vibration motor is used.

In an embodiment of the present application, the sensor assembly is further configured to detect the temperature of the skin surface and whether the skin surface is sufficiently contacted with the surface of the treatment tip. The alarm is further configured to alarm when the sensor assembly detects and determines that the temperature of the skin surface is greater than a preset temperature value or when the sensor assembly detects that the skin surface is sufficiently contacted with the surface of the treatment tip.

In this embodiment, the sensor assembly further includes a contact sensor. The contact sensor detects whether the treatment tip is fully fitted to the skin. If it is not fitted, the controller does not execute the work step of the speed feedback, and the alarm gives an alarm.

The sensor assembly further includes a temperature sensor. When the temperature sensor detects that the temperature of the skin surface is higher than the preset temperature value, stopping the outputting of the ultrasound pulse, and the operator is reminded by the alarm.

When the treatment handle performs treatment, the operator moves the handpiece, keeps the ultrasonic window close to the skin during sliding movement. The ultrasound pulse is outputted in a pulsed manner. The focal positions of the ultrasound pulse are controlled by continuously moving the handpiece to achieve the treatment effect. The ultrasonic treatment transducer inside the treatment tip outputs focused pulse trains, which manifests itself as a heating effect on the tissue, and the heat diffuses from the focus point to its surrounding to form a temperature rising zone/heat diffusion zone.

The heat diffusion zone formed by the ultrasound pulse outputted by the handpiece in a static state is relatively small, and the temperature in the central area is relatively high; the heat diffusion zone formed by the ultrasound pulse outputting during the movement of the handpiece is relatively large, and the temperature in the central area of the heat diffusion zone is lower than the central temperature of the heat diffusion zone formed in the static state of the handpiece, which can effectively reduce the pain. Therefore, the heat diffusion zone formed during the moving of the handpiece has better energy distribution uniformity and thus more comfortable experience.

In an embodiment of the present application, the specific range value of the working parameters of the ultrasonic handpiece is: the output power is in a range of 1 W to 30 W, the output frequency is between 500 kHz and 15 MHz, and the depth of action on the subcutaneous tissue is in a range of 0.5 mm to 25 mm.

In an embodiment of the present application, the specific range value of the working parameters of the handpiece is: a repetition frequency is between 2 Hz and 50 Hz. The output frequency of the ultrasonic handpiece is between 500 kHz and 15 MHz, which is the frequency f1 of the fundamental wave. The repetition frequency of the handpiece in work is the first preset pulse frequency f2; the pulse frequency outputted by the ultrasonic generation source is f1; obtained by frequency modulation, the output frequency when the treatment handle is working is f2.

In an embodiment, the handpiece is moved by the operator during treatment, and moved in a spiral circle trajectory with different focal positions in the treatment area.

In this embodiment, before the handpiece performs the treatment, the modulation method for ultrasonic output pulses presses the skin to ensure the handpiece fully fit with the skin. During the treatment, by controlling the handpiece to move continuously, sliding the treatment tip continuously, the applied force keeps the same during sliding movement, using the same point and moving in a spiral circle trajectory to achieve treatment operations in a small area and small scale, thus achieving the treatment effect.

During treatment, it can also draw circles with different positions as the center in the treatment area, the center of each circle is different and intersecting with each other; finally achieve relatively uniform energy distribution in the entire treatment surface, and move the handpiece to achieve the purpose of treatment. The energy outputted during treatment is relatively uniform, and the temperature superposition effect is better, so as to achieve the treatment effect.

During treatment, it can also control the handpiece to slide back and forth in a Z-shaped trajectory in the treatment area, with uniform force during sliding, and continuously spread the ultrasound pulse by sliding back and forth, and finally spread the treatment energy to all treatment areas to achieve the treatment effect.

The above are only some embodiments of the present application, and are not intended to limit the scope of the present application. Under the concept of the present application, any equivalent structure transformation made by utilizing the description and accompanying drawings of the present application, or directly or indirectly applied in other related technical fields, is included within the scope of the present application.

What is claimed is:

1. A modulation method for ultrasonic output pulses, comprising:
obtaining an output frequency of a first preset pulse, and modulating a to-be-modulated pulse according to the output frequency of the first preset pulse to obtain a first pulse train;
obtaining a period length of a second preset pulse, and dividing the first pulse train into N of the second pulse trains according to the period length of the second preset pulse; and
controlling a transducer to sequentially output the N of the second pulse trains.

2. The modulation method for ultrasonic output pulses according to claim 1, wherein the obtaining the period length of the second preset pulse, and dividing the first pulse train into N of the second pulse trains according to the period length of the second preset pulse further comprises:
obtaining a pulse width of a period of the second preset pulse and an Off time of the period of the second preset pulse; and
determining the period length of the second preset pulse according to the pulse width of the period of the second preset pulse and the Off time of the period of the second preset pulse.

3. The modulation method for ultrasonic output pulses according to claim 2, wherein the pulse width of the period of the second preset pulse is less than a constant time for human pain perception.

4. The modulation method for ultrasonic output pulses according to claim 2, wherein a number of pulse widths of the N of the second preset trains is identical with a number of pulse widths within a first pulse train period.

5. The modulation method for ultrasonic output pulses according to claim 1, further comprising:
adjusting a duty ratio of each of the pulse trains and/or a voltage amplitude of each of the pulse trains to control an output power of each of the pulse trains.

6. The modulation method for ultrasonic output pulses according to claim 5, wherein the adjusting the duty ratio of each of the pulse trains further comprises:
adjusting the pulse width and/or an Off time of each of the pulse trains to control the duty ratio of each of the pulse trains.

7. The modulation method for ultrasonic output pulses according to claim 1, wherein the controlling the transducer to sequentially output the N of the second pulse trains further comprises:
controlling a treatment tip to vibrate slightly along a direction parallel with a skin surface while controlling the transducer to sequentially output the N of the second pulse trains.

8. A controller, comprising a memory and a processor, wherein the memory stores a control program for ultrasonic output pulses, and the modulation method for ultrasonic output pulses according to claim 1 is implemented when the control program for ultrasonic output pulses is executed by the processor.

9. An ultrasonic treatment apparatus, comprising:
a treatment tip provided with a transducer inside;
an ultrasonic generating unit for modulating ultrasonic pulses outputted by the transducer; and
the controller according to claim 8, wherein the controller is connected to the ultrasonic generating unit and configured to control the ultrasonic generating unit to operate to modulate pulse trains outputted by the transducer;
wherein the controller is further configured to control the treatment tip to vibrate slightly along a direction parallel with a skin surface, and control the transducer to output the pulse trains.

10. The ultrasonic treatment apparatus according to claim 9, further comprising:
an ultrasonic handpiece,
wherein the treatment tip is provided at the ultrasonic handpiece; the ultrasonic handpiece comprises a point-type handpiece and a linear-type handpiece; the linear-type handpiece is configured to apply multiple focuses of the ultrasonic energy in line at one shot, being capable of discretely moving at a preset interval along a skin surface of the treatment area; and the point-type handpiece is configured to apply one focus of the ultrasonic energy at one shot, being capable of continuously sliding and overlapping for a preset time along the skin surface of the treatment area.
